(19) United States
(12) Patent Application Publication
(10) Pub. No.: US 2020/0305802 A1
(43) Pub. Date: Oct. 1, 2020

(54) ADAPTING THE DISPLAY OF PATIENT DATA BASED ON DISPLAY SCREEN-REAL ESTATE AND PATIENT MONITORING CONTEXT

(71) Applicant: General Electric Company, Schenectady, NY (US)

(72) Inventors: Francois Archambault, Terrebonne (CA); Abhishek Sahu, Bangalore (IN); Juha Kaasinen, Siilinjärvi (FI); Manoj Kaushik, Bangalore (IN)

(21) Appl. No.: 16/363,337

(22) Filed: Mar. 25, 2019

(51) Int. Cl. A61B 5/00; G16H 10/60

(52) U.S. Cl. CPC A61B 5/742; G16H 40/63; A61B 5/0002; G16H 10/60

(57) ABSTRACT

Techniques are provided for adapting the display of patient data based on display screen-real estate and patient monitoring context. In one or more embodiment, a method is provided that comprises determining, by a system comprising a processor, relative priority of subsets of patient data based on patient and medical provider context, and determining, by the system, an arrangement and sizing scheme for displaying the patient data that maximizes usage of available display screen real-estate based on the relative priority of the subsets. The method further comprises displaying, by the system, the patient data on the available display screen real-estate in accordance with the arrangement and sizing scheme.

### TECHNICAL FIELD

This application generally relates to patient monitoring and more particularly to techniques for adapting the display of patient data based on display screen-real estate and patient monitoring context.

### SUMMARY

The following presents a summary to provide a basic understanding of one or more embodiments of the invention. This summary is not intended to identify key or critical elements or to delineate any scope of the particular embodiments or any scope of the claims. Its sole purpose is to present concepts in a simplified form as a prelude to the more detailed description that is presented later. In one or more embodiments described herein, systems, computer-implemented methods, apparatus and/or computer program products are described that provide techniques for adapting the display of patient data based on display screen-real estate and patient monitoring context.

According to an embodiment, a system can comprise a memory that stores computer executable components and a processor that executes the computer executable components stored in the memory. The computer executable components can comprise a reception component that receives patient data and a prioritization component that determines relative priority of subsets of the patient data to patient and medical provider context. The computer executable components can further comprise a display assessment component that determines an arrangement and sizing scheme for displaying the patient data based the relative priority of the subsets and available display screen real-estate, and a display component that displays the patient data on the available display screen real-estate in accordance with the arrangement and sizing scheme.

In various embodiments, the display assessment component can determine the arrangement and sizing scheme to maximize usage of the available display screen real-estate. For example, in some implementations, the computer executable components further comprise a data grouping component that groups the patient data into the subsets based on defined grouping criteria and wherein the display assessment component further determines the arrangement and sizing scheme based on a number of the subsets. In another example, the display assessment component can determine the arrangement and sizing scheme that spreads the patient data across all of the plurality of display monitors. The display assessment component can also determine the arrangement and sizing scheme by sizing higher priority components of the patient data larger than lower priority components of the patient data.

In one or more embodiments, the arrangement and sizing scheme comprises a first arrangement and sizing scheme, and wherein based on reception of input selecting a subset of the subsets, the display assessment component determines a second arrangement and sizing scheme for displaying one or more components of the subset based on the available display screen real-estate. The display component can further adapt the patient data as displayed via the available display screen real-estate in accordance with the second arrangement and sizing scheme. For example, in some implementations, the first arrangement and sizing scheme comprises a surveillance view comprising patient cards for different patients of a group of monitored patients represented in the patient data, wherein the subset corresponds to a patient card of the patient cards, and wherein the second arrangement and sizing scheme comprises a reduced size representation of the surveillance view on a portion of the display screen real-estate.

In another embodiment, the arrangement and sizing scheme comprises a first arrangement and sizing scheme, and wherein the display assessment component determines a second arrangement and sizing scheme that re-sizes or re-positions a subset of the patient data as displayed in accordance with the first arrangement and sizing scheme based on change in the patient or medical provider context. The display component can further adapt the patient data as displayed via the available display screen real-estate in accordance with the second arrangement and sizing scheme.

In some implementations, the available display screen real-estate can comprise a display of a mobile device and wherein the arrangement and sizing scheme distributes a subset of the patient data for displaying via the display of the mobile device. With these implementations, the display assessment component can determine the subset based on role and privileges of an entity associated with the mobile device.

In one or more additional implementations, the computer executable components can further comprise a machine learning component that learns the patient data relative to patient and medical treater context and builds a data model. For example, the data model can be configured to performs a utility-based analysis that factors cost of incorrectly displaying a subset of patient information relative to benefit of correctly displaying the subset of patient information. In another embodiment, the computer executable components can further comprise an augmented reality component that displays a subset of the patient data as an overlay on eyewear of a medical provider.

In some embodiments, elements described in connection with the system can be embodied in different forms such as a computer-implemented method, a computer program product, or another form.

### DESCRIPTION OF THE DRAWINGS

FIG. 1 illustrates a block diagram of an example, non-limiting system that facilitates adapting the display of patient data based on display screen-real estate and patient monitoring context in accordance with one or more embodiments of the disclosed subject matter.

FIG. 2 illustrates is an example, non-limiting display arrangement for presenting patient data in a surveillance view in accordance with one or more embodiments of the disclosed subject matter.

FIG. 3A illustrates is another example, non-limiting display arrangement for presenting patient data in a surveillance view in accordance with one or more embodiments of the disclosed subject matter.

FIG. 3B illustrates is another example, non-limiting display arrangement for presenting patient data in an inspection view in accordance with one or more embodiments of the disclosed subject matter.

FIG. 3C illustrates an enlarged view of a consolidated representation of a surveillance view configured for display on a single display monitor in accordance with one or more embodiments of the disclosed subject matter.

FIG. 4 illustrates a block diagram of another example, non-limiting system that facilitates adapting the display of patient data based on display screen-real estate and patient monitoring context in accordance with one or more embodiments of the disclosed subject matter.

FIG. 5 illustrates a block diagram of another example, non-limiting system that facilitates adapting the display of patient data based on display screen-real estate and patient monitoring context in accordance with one or more embodiments of the disclosed subject matter.

FIG. 6 illustrates a block diagram of another example, non-limiting system that facilitates adapting the display of patient data based on display screen-real estate and patient monitoring context in accordance with one or more embodiments of the disclosed subject matter.

FIG. 7 provides a flow diagram of an example, non-limiting computer-implemented method for adapting the display of patient data based on display screen-real estate and patient monitoring context in accordance with one or more embodiments of the disclosed subject matter.

FIG. 8 provides a flow diagram of another example, non-limiting computer-implemented method for adapting the display of patient data based on display screen-real estate and patient monitoring context in accordance with one or more embodiments of the disclosed subject matter.

FIG. 9 provides a flow diagram of another example, non-limiting computer-implemented method for adapting the display of patient data based on display screen-real estate and patient monitoring context in accordance with one or more embodiments of the disclosed subject matter.

FIG. 10 illustrates a block diagram of an example, non-limiting operating environment in which one or more embodiments described herein can be facilitated.

### DETAILED DESCRIPTION

The following detailed description is merely illustrative and is not intended to limit embodiments and/or application or uses of embodiments. Furthermore, there is no intention to be bound by any expressed or implied information presented in the preceding Background or Summary sections, or in the Detailed Description section.

Modern healthcare information technology (IT) systems provide virtualized platforms and network-wide compatibility that connect various healthcare data sources, from patient electronic health record systems (EHR), to live patient tracking and monitoring systems, providing access to a plethora of patient and healthcare system information from any connected device. This has enabled many advancements in healthcare, including remote patient monitoring and telemedicine, that enhance care delivery and improve patient outcomes for conditions that need constant surveillance. For example, centralized patient monitoring systems that collect dynamic patient information including monitored vital signs, patient status, patient location within a hospital, patient records and the like, provide a means for caregivers to regularly observe patients in multiple intensive care units (ICUs) from a single remote location. These systems can also provide software tools that support analysis of patient data, vital signs, trends and the like. However, although technological advancements have provided a means to aggregate and access various types of patient information in a centralized network accessible platform, techniques for efficiently and effectively consuming and managing the information are falling behind.

For example, the term “tele-ICU” refers to a system wherein a centralized or remotely based critical care team is networked with the bedside ICU team and patient via state-of-the-art audiovisual communication and computer systems. The tele-ICU team can provide surveillance and support for a large number of ICU patients (e.g., 40-50 patients) in disparate geographical locations for multiple hospitals. As per need, the tele-ICU team can access details of monitored patients and interact with individual patients and/or the bedside team virtually (e.g., via a phone/video call) to provide auxiliary expertise and advise. The technology platform includes various vendor-specific components of hardware and software and affects both the tele-ICU and bedside teams. The tele-ICU team requires the same access as the bedside team to data elements related to patient care (e.g., vital signs, results of laboratory tests, radiologic images, orders, and notes) to assess patients' status accurately and to identify actual and/or potential issues related to patient care.

Given the large number of patients being monitored, the many data elements pertaining to each patient, and the dynamic and critical nature of many of these data elements, the tele-ICU operator needs an efficient and effective mechanism to manage and process the patient data. Many existing tele-ICU systems attempt to accomplish this goal through the use of multiple display monitors to organize and present information for different patients to the tele-ICU operator. However, these systems generally dedicate each monitor to a specific information source or information type. For example, a first monitor may be dedicated to displaying a list of all monitored patients while the other monitors are often dedicated to displaying a specific source or type of information for a single selected patient. For example, a second monitor may be dedicated to displaying radiology images for the selected patient, a third monitor may be dedicated to displaying a live video feed of the selected patient, and the like. With these systems, when a specific patient is not selected, the first monitor displays the patient list while the other monitors are left blank. In addition, the type or source of information for display on dedicated monitory may not be available or relevant to every selected patient. With this setup, screen space is not utilized efficiently or effectively.

The subject disclosure provides systems, computer-implemented methods, apparatus and/or computer program products that facilitate adapting the display of patient data to improve overall patient monitoring and user experience by maximizing usage of available display screen real-estate based on context of one or more patients being monitored and/or an entity to which the patient data is presented. For example, in association with a remote monitoring context (e.g., tele-ICU) wherein patient data is received for a plurality of patients that are currently under the care of a healthcare organization (e.g., a hospital, an assisted living facility, an outpatient clinic, etc.), the disclosed techniques provide a mechanism for organizing and displaying relevant information for the respective patients that optimizes usage of available display screen real-estate to facilitate clinical decision making based on priority of individual patient clinical needs while maintiaing a hierarchical view of the patient data for the entire patient population. In various embodiments, the disclosed techniques maximize the usage of screen space in multiple screen monitors by morphing the patient data and spreading it across all the available monitors in accordance with a logical grouping and sizing scheme determined based on the available display screen real-estate, number of patients being monitored, amount of patient data available, type of patient data available, and the relevance/importance of different components of the patient data (e.g., different patients represented in the patient data and/or different data components associated with a same patient) to one or more patients being monitored and/or the clinician viewing and interacting with the patient data. The arrangement (e.g., layout, groupings, etc.) and sizing scheme of the patient data can be adaptable and scaled to any number of monitors.

For example, in one or more embodiments, the disclosed techniques can provide a surveillance view in which patient cards for all monitored patients are displayed across all the display monitors. The surveillance view can present an organized picture representative of all patients currently being monitored, grouped in a logical manner across the available display screen real-estate. For example, the patient cards can be grouped based on patient priority/critical level, patient location (e.g., grouped by ICU, grouped by hospital location, etc.), patient condition, department, or the like. The patient cards can include a high-level overview of information for each patient, such as information identifying the patient, the condition of the patient, the status of the patient, the time of admittance, the location of the patient, and the like. The size of the respective patient cards and the information included in the respective patient cards can be adaptive based on the number of patients and the available display screen real-estate (e.g., number of display monitors and dimensions of the display monitors). For example, the size of the information cards can increase as the total number of patients being monitored decreases. In some implementations, the patient cards for higher priority (e.g., more critical) patients can be larger and/or more prominent than the patient cards for lower priority patients. The content of the respective patient cards can also be tailored based on priority (e.g., more critical information can be displayed over less critical information) and/or relevance to the patient and/or clinician to which the information is presented.

The disclosed techniques further provide an inspection view that provides for selecting a specific patient to investigate on a deeper level and displaying additional information for the selected patient while maintaining the structure of the surveillance view for the entire patient population in a reference frame or window. For example, in one or more embodiments, based on selection of a patient card from the surveillance view, the entire display can morph into an inspection view wherein additional information for the selected patient can be displayed and distributed across a portion of the available display screen real-estate while another portion of the available display screen real-estate can include a resized representation of the surveillance view. For instance, in an implementation in which several display screens are available, one or more of the display screens can be adapted to present information for only the selected patient (e.g., a live video feed of the patient, image/laboratory studies for the patient, a live view of monitored vital signs for the selected patient, etc.). At the same time a reduced size representation of the surveillance view including all patient cards can be presented on a single display screen. The portions of the display screen real-estate allocated to the selected patient data and the representation of the surveillance view can be based not only on the total amount of display screen real-estate, but also the priority of the selected patient, the amount and type of additional information available for the selected patient, the total number of patients monitored, the priority/severity levels of the other monitored patients, the role and context of the monitoring clinician, and various additional contextual factors. With this technique, the monitoring clinician can analyze detailed information for a specific patient while maintaining a contextual understanding of the selected patient in relation to the entire patient population.

In some embodiments, machine-learning and artificial intelligence can be employed to facilitate determining how to organize and display patient information across available display screen real-estate to optimize clinical decision making based on relevance of the patient information to patient and/or medical provider context. For example, in some implementations, a machine learning component can learn the patient data relative to patient and medical treater context and build one or more data models configured to perform a utility-based analysis that factor costs of incorrectly displaying a subset of patient information relative to benefit of correctly displaying the subset of patient information. The data model can further be applied to determine when and how to display certain subsets of the patient information based on patient and/or medical provider context. For example, the data model can be used to facilitate determining how to resize and/or reposition a subset of the displayed patient information based on change in patient or medical provider context.

As used herein, the term “display screen real-estate” refers to the total display space upon which a defined set of patient data can be presented or displayed. The display screen real-estate can encompass one or more display devices (e.g., display monitors). In one or more embodiments, the available display screen real-estate can also include a mobile device (e.g., a smartphone, a tablet computer, etc.) associated with the monitoring clinician. With these embodiments, the patient information displayed on the mobile device can be tailored or customized as a function of role and privileges of an entity authorized to use the mobile device to view and/or interact with monitored patient data (e.g., an owner of the mobile device). For example, highly sensitive patient information in which an authorized user of the mobile device is authorized to view can be displayed on the mobile device as opposed to a network accessible monitor. In addition to utilizing mobile device displays, in other embodiments, the disclosed techniques can be integrated with augmented reality (AR) and/or virtual reality (VR) systems, wherein patient information can also be displayed via an AR and/or VR display.

Although various embodiments for optimizing the display of patient information are exemplified in the context of remote patient monitoring and tele-ICU, it should be appreciated that the disclosed techniques can be applied in various other healthcare domains. For example, the disclosed techniques can be applied to facilitate optimizing the display of patient information in association with internal (e.g., in facility) management of patients admitted to a same healthcare facility (e.g., a hospital, an assisted living facility, etc.), a same department of the healthcare facility (e.g., emergency, surgery, recovery, etc.). In another example, the disclosed techniques can be applied to facilitate optimizing the display of healthcare facility information regarding utilization of medical resources (e.g., beds, medical supplies, medical equipment, medical personnel, etc.), to facilitate managing and optimizing clinical and/or financial performance of the healthcare facility.

As used herein, the term “clinician” can refer to any individual or entity that is involved in the treatment and observation of living patients, as distinguished from one engaged in research. For example, a clinician can include but not limited to: a physician or doctor, a physicians' assistant, a nurse, a nurse practitioner, a midwife, a medical technician, a remote monitoring observer, one or more individuals involved in patient ICU care (e.g., onsite and/or remotely) and the like. Further in some embodiments, a machine (e.g., a robot, an IMD, medical equipment, medical devices, etc.) can be configured to perform a medical procedure or provide medical treatment. Thus, in some embodiments, the term “clinician” can also encompass a machine that can perform a medical procedure or provide medical treatment. The term “healthcare organization” is used herein to refer to any entity (e.g., including a company, a group of individuals, or a solo individual) that provides healthcare services to patients, whether it be at a designated healthcare facility, in an ambulatory vehicle, or out in the field. The terms healthcare facility or medical facility as used herein can refer to any physical location (e.g., building, building complex, campus, etc.) where healthcare is provided. Healthcare facilities can range from small clinics and doctor's offices to urgent care centers and large hospitals with elaborate emergency rooms and trauma centers. Various embodiments of the subject disclosure can be applied in association with managing and optimizing healthcare at a wide range of healthcare facilities, from large and complex hospitals that are equipped to perform a wide range of medical procedures to small town clinics. It should be appreciated that the disclosed techniques can be employed to facilitate provision of medical care to a patient in any scenario that involves a human (e.g., one or more clinicians, a non-clinician in emergency situations and even the patient himself/herself) providing the care. In addition, in some embodiments, a machine (e.g., and IMD, a robot, etc.) can be configured to perform medical procedures. Thus, in some embodiment, the disclosed techniques can further be applied to monitor and optimize provision of medical care by machines.

One or more embodiments are now described with reference to the drawings, wherein like referenced numerals are used to refer to like elements throughout. In the following description, for purposes of explanation, numerous specific details are set forth in order to provide a more thorough understanding of the one or more embodiments. It is evident, however, in various cases, that the one or more embodiments can be practiced without these specific details.

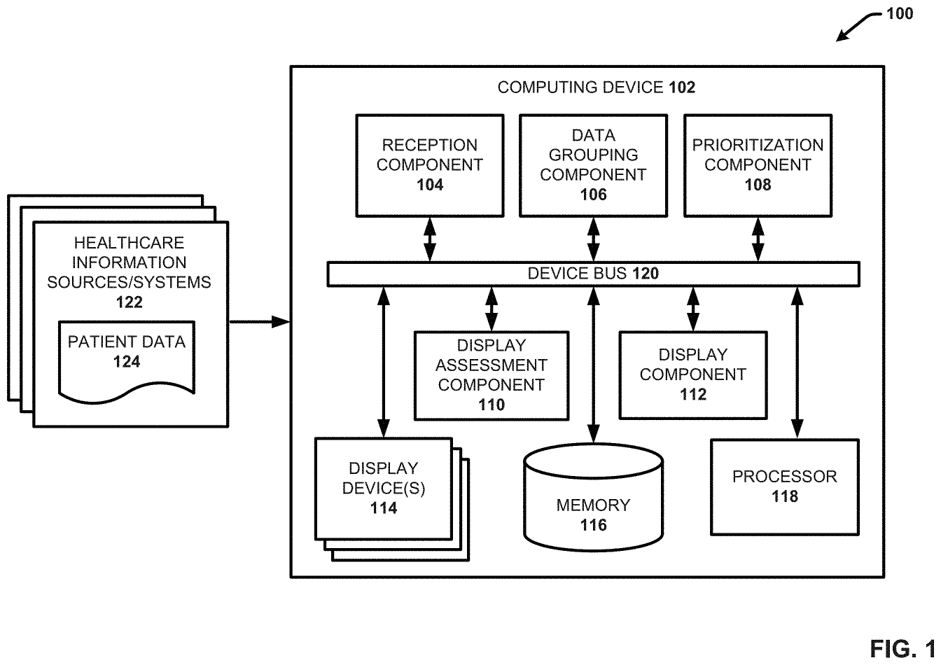

Turning now to the drawings, FIG. 1 illustrates a block diagram of an example, non-limiting system 100 that facilitates adapting the display of patient data based on display screen-real estate and patient monitoring context in accordance with one or more embodiments of the disclosed subject matter. Embodiments of systems described herein can include one or more machine-executable components embodied within one or more machines (e.g., embodied in one or more computer-readable storage media associated with one or more machines). Such components, when executed by the one or more machines (e.g., processors, computers, computing devices, virtual machines, etc.) can cause the one or more machines to perform the operations described.

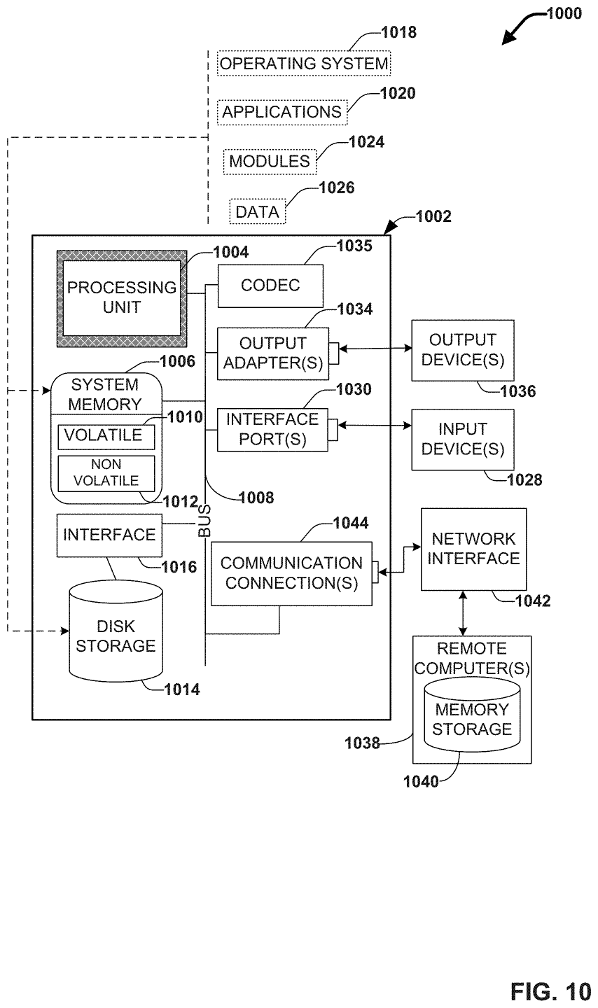

System 100 includes one or more healthcare information sources/systems 122 and a computing device 102. Generally, the computing device 102 can include or be operatively coupled to at least one memory 116 that stores computer executable components and at least one processor 118 that executes the computer executable components stored in the memory, examples of which can be found with reference to FIG. 18. For example, these computer executable components can include for example, a reception component 104, a data grouping component 106, a prioritization component 108, a display assessment component 110 and display component 112. The computing device 102 can also include or be operatively coupled to one or more display devices 114 configured to present information (e.g., patient data 124) in a visual or tactile form. For example, the one or more display devices 114 can include one or more computer monitors, one or more television screens, one or more electronic image projection devices or the like. The computing device 102 can also include a device bus 120 to communicatively couple the various components of the computing device 102 (e.g., the reception component 104, the prioritization component 108, the display assessment component 110, the display component 112, the one or more display devices 114, the memory 116 and the processor 118). Examples of said processor 118 and memory 116, as well as other suitable computer or computing-based elements, can be found with reference to FIG. 10, and can be used in connection with implementing one or more of the systems or components shown and described in connection with FIG. 1 or other figures disclosed herein.

The various components and devices of system 100 can be connected either directly or via one or more networks, (not shown). Such network(s) can include wired and wireless networks, including but not limited to, a cellular network, a wide area network (WAN, e.g., the Internet), a local area network (LAN), or a personal area network (PAN). For example, the computing device 102 can communicate with the one or more healthcare information sources/systems, (and vice versa), using virtually any desired wired or wireless technology, including, for example, cellular, WAN, wireless fidelity (Wi-Fi), Wi-Max, WLAN, etc.

In accordance with various aspects and embodiments described herein, the computing device 102 can be configured to facilitate displaying the patient data 124 received from one or more healthcare information sources/systems 122 with an adaptive arrangement and sizing scheme that facilitates efficient and effective clinical evaluation of the patient data 124 based in part on available display screen real-estate provided by the one or more display devices 114. For example, in association with a remote monitoring context (e.g., tele-ICU) wherein patient data 124 is received for a plurality of patients that are currently under the care of a healthcare organization (e.g., a hospital, an assisted living facility, an outpatient clinic, etc.), the computing device 102 can be configured to organize and display relevant information for the respective patients in a manner that optimizes usage of available display screen real-estate to remotely facilitate their clinical care based on priority of individual patient clinical needs while maintaining a hierarchical view of the entire patient population.

In this regard, the computing device 102 can include reception component 104 to receive and/or retrieve the patient data 124 from the one or more healthcare information sources/systems 122. The one or more healthcare information sources/systems 122 can include internal systems and databases associated with one or more healthcare organizations, as well as systems and database that are openly accessible to public entities. The healthcare information sources/systems 122 can provide a variety of patient data 124 for one or more patients associated with one or more healthcare organizations that can facilitate evaluating and monitoring provision of healthcare to the respective patients. The patient data 124 can include static or relatively static information for respective patients (e.g., historical health records, medical images, medical laboratory study information, etc.) as well as dynamic information that can be generated and/or received in real-time or substantially real-time (e.g., monitored vital signs, tracked events that occur over the course of care, patient location, live audio/visual feeds, etc.).

For example, the patient data 124 can include electronic medical record (EMR) and/or electronic health record (EHR) data for patients provided by one or more EMR/EHR healthcare sources/systems. The EMR/EHR data can include known and recorded medical and/or health information about current and past patients of one or more healthcare organizations. In this regard, the EMR/EHR data can include information traditionally found in patient EMR or EHRs including but not limited to: information identifying patients, information providing demographic profile information for respective patients, information regarding past medical history (e.g., including past diagnosis, procedures, conditions, etc.), physician reports/nots, medication and allergy information, immunization dates, laboratory report data, imaging studies, pathology report data, care plan information, insurance information, and the like.

The patient data 124 can also include dynamic information related to a patient's current physiological state or condition, course of care, location, mobility state, status (e.g., stable, critical, etc.), and the like. For example, in some implementations, the patient data 124 can include information received in association with initiation of treatment of a patient that provides an assessment of a condition or diagnosis of a patient for which medical care is being provided to the patient. The patient data 124 can further include any information gathered over the course of care relating to the physiological state or condition of the patient. For example, the patient data 124 can include information regarding tracked physiological parameters of a patient (e.g., vital signs and various other physiological parameters), timing associated with the tracked physiological parameters (e.g., onset, duration, etc.), changes in a physiological status or state of a patient, changes in appearance of the patient, physical range of motion or movements capable of being performed by the patient, expressed symptoms and side effects of the patient, expressed levels of pain experienced by the patient, information regarding a mental state of the patient, and the like.

The specific physiological parameters that are tracked can be predefined based on a condition or the patient or course of care for the patient. For example, the physiological data that is relevant to a patient undergoing a course of labor can be different to those of a patient receiving a heart transplant. Accordingly, the specific patient data that is collected and reported in real-time can be tailored to the specific context of a patient's condition and treatment. For example, for labor patients, there are key data and decision points that can affect the outcome of the baby including lifelong cognitive and physical functions. For instance, these key data points can include but are not limited to: timing of onset of labor, timing of decision to induce, timing of the active phase, timing of rupture of the membrane, timing and dosage of certain administered pharmaceuticals, changes in dilation, fetal heart rate, fetal deceleration, maternal temperature, maternal hypertension, timing of hypoxia onset, and the like. According to this example, for labor patients (or other types of patients grouped by medical diagnosis or procedure), the patient data 124 can include information corresponding to or related to these key data point.

The patient data 124 can also include real-time information regarding the clinical workflow followed for a patient over the course of care, including clinical events that occur over the course of care. In this regard, the patient data 124 can identify and describe medical treatment provided to a patient by one or more clinicians over the course of care. This can include but is not limited to: procedures performed, specific steps of the procedures performed, relevant aspects/parameters associated with respective steps, timing and order of the steps, medical instruments/supplies used, clinicians involved in performance of procedural steps, roles and actions performed by the respective clinicians, medications administered, dosage of the medications administered, timing of patient check-ups, and the like. The specific workflow events that are tracked and reported in-real time can vary based on the clinical context of each patient. The patient data 124 can also include information regarding upcoming scheduled medical treatment, (e.g., type of scheduled treatment, clinician/physician involved, timing/duration associated with the treatment, etc.).

In some implementations, the patient data 124 can also identify or track a current location of a patient relative to a healthcare facility (e.g., in an ambulance on the way to the emergency room, within a specific unit of the healthcare facility, within a specific room of the healthcare facility, etc.). The patient data 124 can also include information regarding current and/or scheduled medical treatment or procedures of a patient. The patient data 124 can also include live audio/visual data captured of a patient in association with admittance and/or provision of medical treatment to the patient.

The types of healthcare information sources/systems 122 from which dynamic types of patient data 124 is received can vary. In some implementations, the patient data 124 can be received directly from one or more medical monitoring devices associated with the patient and configured to read and report monitored physiological parameters. For example, the medical monitoring devices can include a device worn by, within (e.g., an implantable medical device (IMD)), and/or physically connected to the patient and configured to capture and/or monitor physiological information (e.g., physiological parameters) that can be sent to or otherwise received by the computing device 102 in real-time or substantially real-time. In other implementations, the patient data 124 can include information observed by or determined by a clinician and entered into an electronic tracking system that records and reports observed changes in physiological states of a patient. For example, the electronic tracking system can receive information observed and/or determined by a first responder, paramedic, nurse, physician, etc., regarding the physiological state or condition of the patient over the course of care. In another example, the patient data 124 can include information entered into a healthcare management system, such a bed assignment management system, a diagnosis/reporting system, a course of care tracker, a procedures scheduling system, and the like. In another example, the patient data 124 can be received from an electronic symptom tracking system that receives information entered by the patient (or an authorized agent of the patient) regarding self-reported medical symptoms or other information about how the patient is feeling mentally and/or physically (e.g., level of pain experienced, level of energy, stress level, mood information, etc.).

In one or more embodiments, the specific type of patient data 124 that is received by the computing device 102 can vary based on the intended use of the patient data 124. For example, the specific type of patient data 124 can vary based on whether the computing device 102 is configured to process the patient data 124 for the purpose of patient monitoring, diagnosis evaluation, performance evaluation, live care assistance, patient flow management, and the like. For example, with respect to tele-ICU, the patient data 124 that is received or retrieved by the reception component 104 can include information identifying respective patients included in different ICUs, EMR/EHR data for the respective patients, dynamic information regarding current status of the respective patients, monitored vital signs, live audio/visual data of the patient, and the like. In this regard, depending on the purpose of evaluation of the received patient data 124, the reception component 104 can receive patient data 124 comprising predefined data elements or data components. For example, in some embodiments, the patient data 124 can include defined subsets of patient data for each patient being monitored, wherein patient data associated with a single patient can be grouped as a single group or subset of the patient data, and wherein each subset (e.g., one for each patient) can include defined data elements or components pertaining to the monitored patient (e.g., an EMR/EHR component, a vital signs component, a live video feed component, etc.).

In the embodiment shown, the computing device 102 can include data grouping component 106 to facilitate grouping the received patient data 124 into logical groups or subsets. In some implementations, the data grouping component 106 can be configured to employ predefined grouping criteria (e.g., a data model) to group the patient data 124 into different groups or subsets. For example, the predefined grouping criteria can include grouping by patient (e.g., wherein data associated with a single patient is grouped together in a single subset), grouping by diagnosis, grouping by procedure code, grouping by location (e.g., hospital, department, unit within a department, etc.), grouping by level of severity or urgency of care (e.g., critical patients vs. non-critical patient), grouping by treating physician, grouping by time of admittance, etc. In some embodiments, the data grouping component 106 can generate a plurality of different groupings of the patient data, including subgroups within groups. In this regard, the data grouping component 106 can determine logical connections between components of the patient data 124 grouped be one or more defined criteria. In some embodiments, (discussed in greater detail infra), the data grouping component 106 can employ one or more machine learning techniques to identify and characterize relationships between different components of the patient data 124 to facilitate grouping the patient data 124 into different logical sets or subsets.

In various embodiments, the prioritization component 108 can evaluate the received patient data 124 to determine the relative priority of different components, sets/groups or subsets/subgroups of the patient data 124 to patient and/or medical provider context, wherein the relative priority of the different components, sets or subsets of the patient data 124 can control the arrangement and sizing scheme for displaying the patient data. In this regard, in one or more embodiments, the display assessment component 110 can determine an arrangement and sizing scheme for displaying the patient data 124 based in part on the relative priority of the components, sets or subsets of the patient data and the available display screen real-estate (e.g., provided by the one or more display devices 114). The display component 112 can further display the patient data on the available display screen real-estate (e.g., on the one or more display devices 114) in accordance with the arrangement and sizing scheme determined by the display assessment component 110.

In one or more embodiments, the prioritization component 108 can determine priority scores or values representative of a level of importance/relevance of the grouped components of the patient data 124, and the display assessment component 110 can determine how to organize, arrange and display the patient data (e.g., determine what components of the patient data to display when, how to structure and size the patient data, etc.), based on the priority scores or values associated therewith. The prioritization component 108 can employ various prioritization criteria to determine relative priority of different subsets or components of the patient data 124 to a particular context in which the patient data 124 is used (e.g., one or more goals of the entity reviewing and evaluating the patient data 124). For example, different data elements included in the patient data can be more or less relevant based on a usage context of the patient data 124, such as (but not limited to), patient monitoring, diagnosis evaluation, performance evaluation, live care assistance, patient flow management, quality control (e.g., identify errors or deficiencies in provision of medical treatment relative to a defined protocol), and the like. In this regard, different subsets and/or components of the patient data 124 can be more or less relevant to the reviewing entity based on the usage context. Thus, the prioritization criteria and/or scheme employed by the prioritization component can be tailored to account for the purpose in which the patient data is being evaluated.

For example, with respect to remote monitoring and tele-ICU in which many different patients are monitored simultaneously, the prioritization component 108 can score the respective patients with priority information reflective of their relative need focus and attention relative to other patients. For instance, in accordance with usage of the patient data for tele-ICU, the prioritization component 108 can determine priority scores for the monitored patients reflective of their priority level relative to one another and/or the reviewing entity based on one or more factors including but not limited to: severity or critical nature of the patient's condition, status of the patient, change in vital signs monitored, comorbidity of the patient, age of the patient, time of admittance to the ICU, and the like.

The prioritization component 108 can also determine relative priority levels of different components of the patient data 124 associated with a single patient based on the purpose of the entity reviewing the patient data. For example, with respect to tele-ICU, various different types of data can be received for a monitored patient, such as data elements associated with the patient's EMR/HER, laboratory data, medical images, monitored physiological data, tracked events over the course of care, and the like. In accordance with this example, the prioritization component 108 can determine the priority of the different data components associated with a specific patient relative to a current context of the patient and/or the entity reviewing the patient. For example, stable vital signs data can be associated with a lower priority than results of a recent imaging study indicating a potential critical diagnosis requiring urgent attention. However, a sudden change in the patient context such as a sudden change in the vital signs of the patient can result in re-prioritization of the patient's data. For instance, reception of information regarding a sudden change in the patient's vital signs can be given higher priority than other information for the patient. The relative priority of different components of patient data associated with a single patient can also be based on a condition of the patient, medical history of the patient, demographics of the patient, a procedure performed, a current event in the patient's course of care, etc.

In some embodiments, the prioritization component 108 can also prioritize components of the patient data 124 in relation to a known role, skill, and/or preference or the reviewer. For example, if the reviewer has an expertise in radiology, imaging data can be prioritized higher than laboratory data. The prioritization component 108 can also employ learned user preferences (e.g., learned using machine learning, as discussed in greater detail infra) regarding user interaction with the data as presented to the data via the display component 112 to determine data elements that more are considered more important/relevant to the reviewer based on patient and data usage context.

In some embodiments, the prioritization component 108 can prioritize the patient data 124 after the patient data has been grouped into logical groups by the data grouping component 106. In other embodiments, the prioritization component 108 can facilitate grouping the patient data 124 into one or more subsets based on priority of one or more components of the patient data to patient and/or medical provider context. For example, in some implementations, the prioritization component 108 can determine priority scores for respective patients included in a group of evaluated/monitored patients based on severity, timing of admittance, timing of procedure completion, age, demographics, comorbidity, vital sign values, etc., and the data grouping component 106 can group the patients into priority groups based on their respective priority scores.

The display assessment component 110 can employ various techniques to determine the arrangement and sizing scheme for displaying the patient data 124 based on the available display screen real-estate and the relative priority of the different components, groups/sets or subgroups/subsets of the patient data to the purpose or role of the entity to which the patient data is presented. In various embodiments, the display assessment component 110 can determine the arrangement and sizing scheme to maximize usage of the available display screen real-estate in view of the relative priority scores/values (representative of contextual relevance to the reviewing entity) determined for different groups, subgroups and/or components of the patient data 124 and the number/amount of the different groups, subgroups and/or components. For instance, the available display screen real-estate can comprise a plurality of display devices 114 and the display assessment component 110 can determine the arrangement and sizing scheme based on number and dimensions of the display monitors such that higher priority data groups/subgroups are designated a greater area of the display screen real-estate than lower priority subsets. The display assessment component 110 can also determine the arrangement and sizing scheme by sizing higher priority data components of components larger than lower priority data components (e.g., using different sized graphical elements, windows, etc.). The display assessment component 110 can also determine relative locations for displaying different groups, subgroups and/or components of the patient data 124 based on their relative priority scores. For example, the display assessment component 110 can display higher priority data components in more prominent areas of the display screen real-estate (e.g., on a particular display device or area of one or more display devices) relative to lower priority data components. In another example, the display assessment component 110 can display higher priority data components in a central region of the display screen real-estate and display lower priority data components in peripheral regions of the display screen real-estate. In another example, the display assessment component 110 can determine the arrangement and sizing scheme that spreads data elements representative of all monitored patients across all of the plurality of display monitors in accordance with their groupings and container sizes. In some implementations, based on the type of patient data for available for display and the available display screen-real estate, the display assessment component 110 can determine/select what portions (e.g., groups, subgroups and/or components) of the patient data 124 to include in the display based on relative priority (e.g., provided by the prioritization scores associated therewith) to the reviewing entity and/or the patient in association with a current usage context (e.g., goal of the reviewing entity).

The display assessment component 110 can further facilitate adapting the manner in which the patient data 124 is displayed across the available display screen real-estate based on user interaction with the patient data, reception of new patient data and/or changes in dynamic patient data (e.g., changes in vital signs, occurrence of new events or conditions), and corresponding changes in priority scores/levels associated with the new/changed patient data and/or data elements. For example, in some embodiments the display assessment component 110 can determine a first arrangement and sizing scheme for displaying the patient data 124 that maximizes the available display screen real-estate and the display component 112 can render the patient data in accordance with the first arrangement and sizing scheme. The display assessment component 110 can further determine a second arrangement and sizing scheme that re-sizes or re-positions a subset of the patient data as displayed in accordance with the first arrangement and sizing scheme based on change in the patient and/or medical provider context, wherein change in patient and/or medical provider context results in reprioritization of the patient data (e.g., by the prioritization component 108). The display component 112 can further adapt the patient data as displayed via the available display screen real-estate (e.g., as displayed via the one or more display device 114) in accordance with the second arrangement and sizing scheme.

In another example, the rendered patient data can include interactive graphical elements that can be interacted with (e.g., selected and viewed, changed, etc.) in accordance with various graphical user interface (GUI) standards. In this regard, based on received user input selecting a component of the displayed patient data, display assessment component 110 can be configured to determine a new arrangement and sizing scheme for displaying one or more sub-components of the selected component based on the available display screen real-estate. The display component 112 can further adapt the patient data as displayed via the available display screen real-estate in accordance with the second arrangement and sizing scheme. For example, in one or more embodiments the first arrangement and sizing scheme can comprise a surveillance view comprising patient cards for different patients of a group of monitored patients represented in the patient data 124, wherein the selected component corresponds to a patient card of the displayed patient cards. In accordance with this example, the second arrangement and sizing scheme can include patient data related to the second patient provided in first portion of the display screen real-estate. The second arrangement and sizing scheme can further include a reduced size representation of the surveillance view provided in a second portion of the display screen real-estate, wherein the first portion is larger than the second portion, wherein the first portion has a more prominent position relative to the second portion, or the like.

In this regard, in accordance with one or more implementations in which system 100 is used to monitor data associated with a plurality of different patients, the display component 112 can be configured to display a surveillance view in which patient cards for all monitored patients are displayed across the entirety of the available display screen real-estate. The surveillance view can present an organized picture representative of all patients currently being monitored. In some implementations, the patient cards can be grouped in accordance with a grouping scheme defined by the data grouping component 106. For example, the patient cards can be grouped based on patient priority/critical level, patient location (e.g., grouped by ICU, grouped by hospital location, etc.), patient condition, department, or the like. The patient cards can include a high-level overview of information for each patient, such as information identifying the patient, the condition of the patient, the status of the patient, the time of admittance, the location of the patient, and the like. The display assessment component 110 can further determine the size and arrangement of the respective patient cards and the information included in the respective patient cards based on the number of patients and the available display screen real-estate (e.g., number of display monitors and dimensions of the display monitors). For example, the display assessment component 110 can increase the size of the information cards as the total number of patients being monitored decreases. In some implementations, the display assessment component 110 can size patient cards for higher priority (e.g., more critical) patients larger and/or in a more prominent manner (e.g., location, appearance, etc.) than the patient cards for lower priority patients. The display assessment component 110 can also tailor the content presented in the respective patient cards based on priority (e.g., more critical information can be displayed over less critical information) and/or relevance to the patient and/or clinician to which the information is presented.

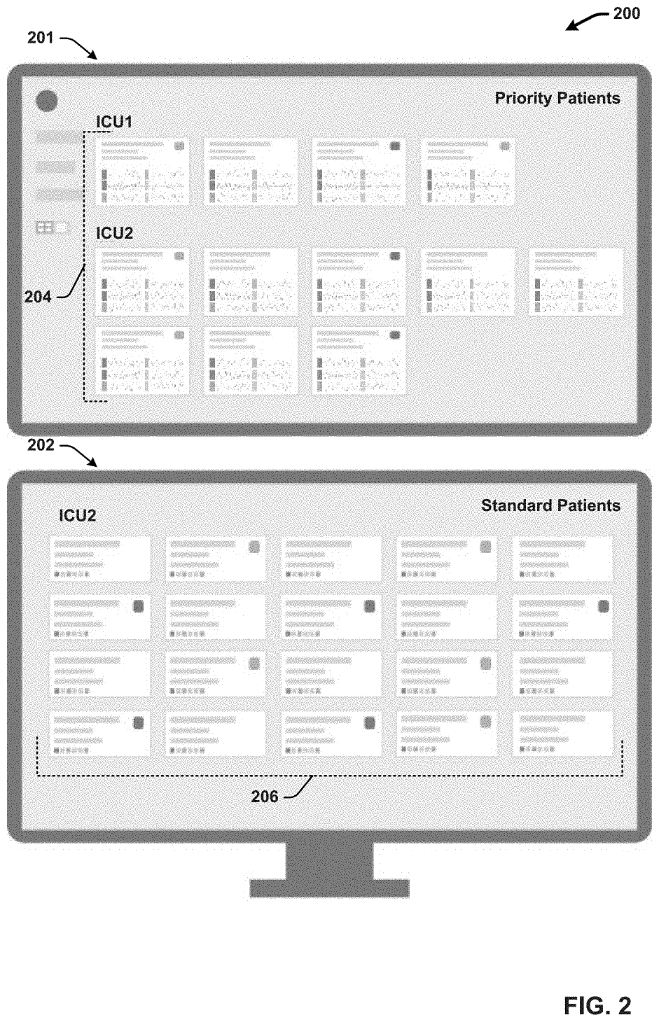

FIG. 2 illustrates is an example, non-limiting display arrangement 200 for presenting patient data in a surveillance view in accordance with one or more embodiments of the disclosed subject matter. In the embodiment shown, the available display screen real-estate comprises two monitors, monitor 201 and monitor 202. The surveillance view includes patient cards (e.g., patient cards 204 and patient cards 206) for each of a plurality of different patients monitored in accordance with a tele-ICU usage context. The display arrangement 200 spreads the totality of the patient cards across both monitors, thereby maximizing usage of the entire display screen real-estate. In addition, the display arrangement 200 configures the size and arrangement of the patient cards based on relative priority of the respective patients associated with the patient cards. In particular, the display arrangement 200 allocates monitor 201 to priority patients and monitor 202 to standard (e.g., lower priority/less critical) patients, thereby visually distinguishing the respective patient groups. The display arrangement 200 further groups the patient cards based on the particular ICU that where the patients are located (e.g., ICU1 or ICU2). In addition, the patient cards 204 for the priority patients are larger than the patient cards 206 for the standard patients. The patient cards 204 for the priority patients also include different (e.g., a greater amount and/or more detailed) information relative to the patient cards 206 for the standard patients.

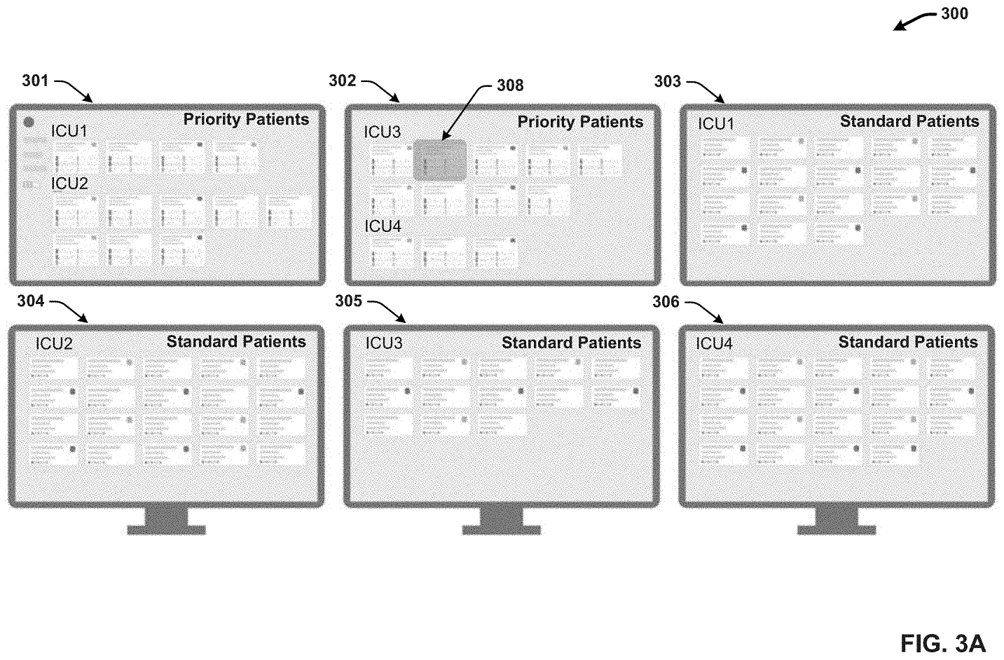

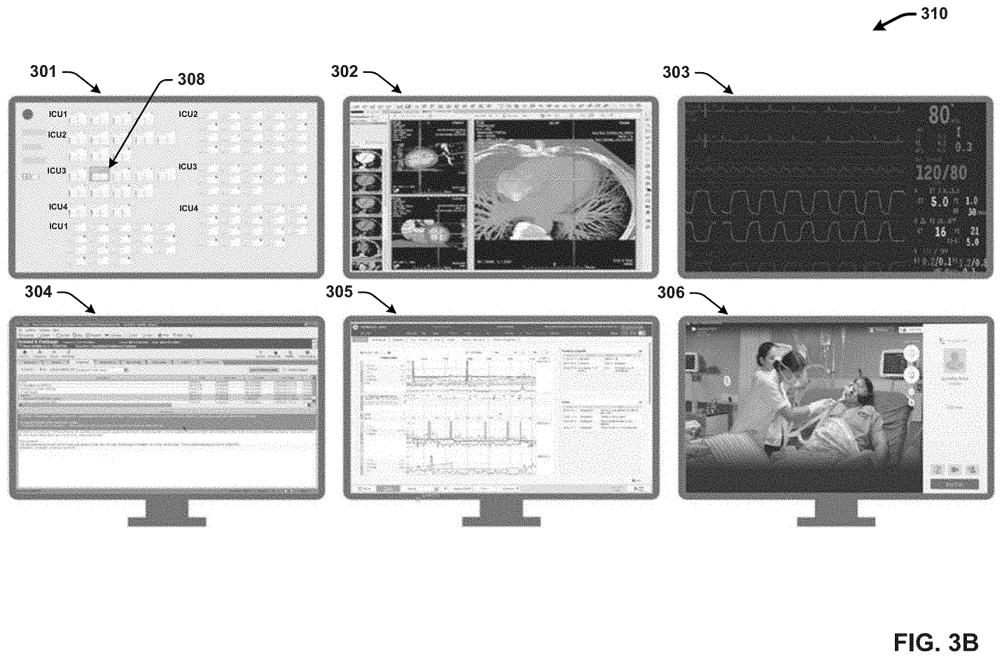

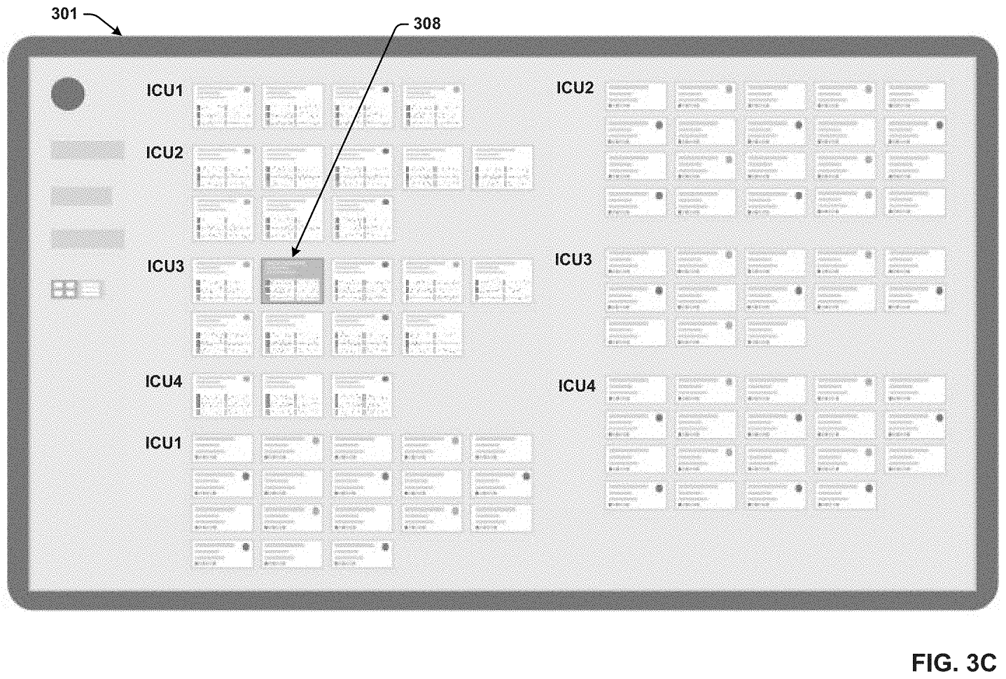

FIG. 3A illustrates is another example, non-limiting display arrangement 300 for presenting patient data in a surveillance view in accordance with one or more embodiments of the disclosed subject matter. Display arrangement 300 demonstrates an example tele-ICU surveillance view in which the available display screen real-estate includes six display monitors (e.g., monitor 301, monitor 302, monitor 303, monitor 304, monitor 305, and monitor 306). Display arrangement 300 provides same or similar configuration features as display arrangement 200 with the addition of more monitors and more patients (and corresponding patient cards). In this regard, similar to display arrangement 200, the display arrangement 300 uses the entirety of the available display screen real-estate, spreading all of the patient cards across all available monitors with a sizing and arrangement scheme that is based on grouping of the patients by ICU location and relative patient priority. Repetitive description of like elements employed in respective embodiments is omitted for sake of brevity.

With reference again briefly to FIG. 1 in view of FIG. 3A, in one or more embodiments, in association with processing of the patient data 124 for patient monitoring (e.g., tele-ICU), the display assessment component 110 can also be configured to provide an inspection view that provides for selecting a specific patient to investigate on a deeper level. For example, in various embodiments, the patient cards rendered in the surveillance view can be selected, wherein in response to reception of user input selection a patient card, the display assessment component 110 can transition to rendering the inspection view. The inspection view can comprise additional information for the selected patient (corresponding to the selected patient card), while also maintaining the structure of the surveillance view for the entire patient population in a reference frame or window. For example, in one or more embodiments, based on selection of a patient card from the surveillance view, display assessment component 110 can adapt the entire display into an inspection view wherein additional information for the selected patient can be displayed and distributed across a portion (e.g., a majority) of the available display screen real-estate while another portion of the available display screen real-estate can include a resized (smaller) representation of the surveillance view.

For example, FIG. 3B illustrates is an example, non-limiting display arrangement 310 for presenting patient data in an inspection view in accordance with one or more embodiments of the disclosed subject matter. Display arrangement 310 provides an example inspection view that can be generated in response to selection of patient card 308 from the (surveillance view) display arrangement 300 shown in FIG. 3A. In accordance with this example implementation, the inspection view optimizes usage of the available display screen real-estate by distributing different data components associated with the selected patient across a majority of the display area (e.g., five out of the six display monitors). For example, monitor 302 comprises medical image data for the selected patient, monitor 303 comprises live vital sign data for the selected patient, monitor 304 comprises EMR/EHR data for the selected patient, monitor 305 comprises laboratory report data for the selected patient, and monitor 306 comprises a live audio/video conferencing feed for the selected patient. At the same time a reduced size representation of the surveillance view including all patient cards can be presented on a single monitor (e.g., monitor 301). FIG. 3C presents an enlarged view of the consolidated representation of the surveillance view displayed on monitor 301 in accordance with display arrangement 310. With this configuration, the monitoring clinician can analyze detailed information for a specific patient while maintaining a contextual understanding of the selected patient in relation to the entire patient population.

In various embodiments, the portions of the display screen real-estate allocated to the selected patient data and the representation of the surveillance view in the inspection view can be based not only on the total amount of display screen real-estate, but also the priority of the selected patient, the amount and type of additional information available for the selected patient, the total number of patients monitored, the priority/severity levels of the other monitored patients, the role and context of the monitoring clinician, and various additional contextual factors. In this regard, it should be appreciated that the specific data components shown in the respective monitors of display arrangement 310 are merely exemplary and can vary based on patient and/or reviewer context (e.g., relevance to the context), available patient data, and the like. In addition, although each monitor comprises includes a different data component, in various implementations, one or more monitors can include a plurality of windows with different data components (e.g., two or more data components can be split across a single monitor).

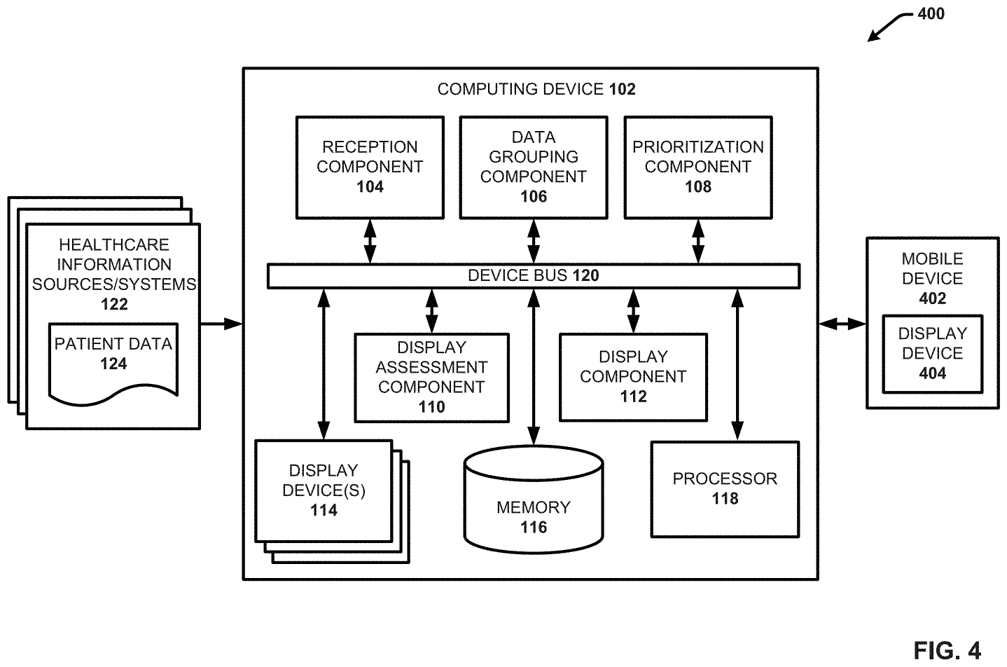

FIG. 4 illustrates a block diagram of another example, non-limiting system 400 that facilitates adapting the display of patient data based on display screen-real estate and patient monitoring context in accordance with one or more embodiments of the disclosed subject matter. System 400 includes same or similar features as system 100 with the addition of mobile device 402. The mobile device 402 can include various suitable computing devices including a display device 404. For example, the mobile device can include but is not limited to, a laptop computer, a mobile phone, a smartphone, a tablet personal computer (PC), a personal digital assistant (PDA), a heads-up display (HUD), an augmented reality (AR) device, a virtual reality (VR) device, a wearable device, and the like. Repetitive description of like elements employed in respective embodiments is omitted for sake of brevity.

In one or more embodiments, a mobile device 402 comprising a display device 404 can be communicatively coupled to the computing device 102, and the display device 404 can serve as an additional display device for displaying the patient data 124. In this regard, the display device 404 can extend the available display screen real estate of the one or more display devices 114. In some implementations of these embodiments, the display assessment component 110 can tailor the portion of the patient data 124 for display on the display device 404 as a function of a role, rights/privileges, and/or preference of an entity associated with the mobile device 402. For example, the mobile device 402 can include a mobile device associated with a known user (e.g., an owner of the mobile device or another authorized user of the mobile device 402), such as the entity reviewing or otherwise having access to the patient data 124 as rendered via the one or more display devices 114. For example, the mobile device 402 can be operated by a reviewing physician or clinician of one or more monitored patient, an authorized agent of one or more monitored patients, or the like.

In some implementations, the role of the user of the mobile device 402 can be based on a clinical specialty of the user. For instance, the user may be a radiology specialist, a pathologist, or the like. With these implementations, the display assessment component 110 can tailor the patient data displayed on the device display to the specialty of the user. For example, the display assessment component 110 can send radiology images to the mobile device 402 for display on the device display. In another implementation, the user of the mobile device 402 can be associated with authorization information that defines or indicates specific portions of the patient data 124 that the user has privileged access to. For example, the authorization information can authorize the user to view sensitive information for one or more identified patients (e.g., personal patients or family members of the user), patients having a specific condition, or the like. With these implementations, the display assessment component 110 can tailor the information that is displayed on the display device 404 to include data that the user of the mobile device has privileged access to. Still in another implementation, the display assessment component 110 can employ preference information regarding preferences of the user of the mobile device regarding what time of data the user prefers to view on the device display as opposed to the one or more display devices 114, and vice versa. With these implementations, the display assessment component 110 can configure the patient data 124 for distributing across the one or more display device 114 and the display device 404 according to the user preference. In some embodiments, the preference information can be determined and/or inferred using machine learning.

In some embodiments, the mobile device 402 can facilitate reviewing and monitoring the patient data 124 by a user at times when the user is not located within viewing proximity of the one or more display device 114. For example, the review may need to step away from the location of one or more display device 114. With these embodiments, the display assessment component 110 can be configured to stream or cast the patient data to the display device based on the location of the mobile device 402. For example, in one embodiment, the display assessment component can generate a mirrored representation of the patient data 124 as displayed via the one or more display device 114 for rendering on the display device 404 based on movement of the mobile device outside of a defined distance from the one or more display devices.

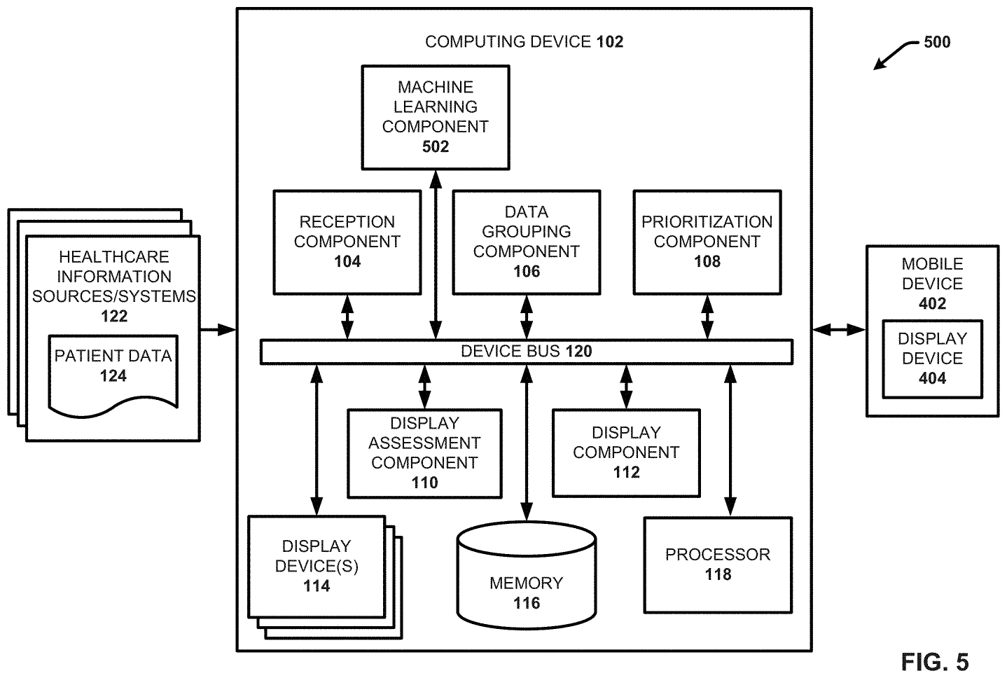

FIG. 5 illustrates a block diagram of another example, non-limiting system 500 that facilitates adapting the display of patient data based on display screen-real estate and patient monitoring context in accordance with one or more embodiments of the disclosed subject matter. System 500 includes same or similar features as system 400 with the addition of machine learning component 502. Repetitive description of like elements employed in respective embodiments is omitted for sake of brevity.

As described noted above, in one or more embodiments, the data grouping component 106, the prioritization component 108 and/or the display assessment component 110 can employ machine learning and/or artificial intelligence to facilitate determining how to group and/or prioritize the patient data 124, and/or configure the patient data 124 for display. In accordance with these embodiments, the computing device 102 can include machine learning component 502 to facilitate learning how to group patient data, prioritize patient data, and/or display patient data in a manner that optimizes user interaction and/or consumption of the patient data in association with achieving a defined goal of the user (e.g., remote patient monitoring, performance evaluation, diagnosis, etc.) while enhancing patient clinical outcomes. In one or more embodiments, the machine learning component 502 can learn the patient data relative to patient and medical treater context and builds a data model configured to determine relevance and/or priority of different components of the patient data to treater context. The machine learning component 502 can also employ one or more machine learning techniques to learn user interaction with different display configuration schemes of patient data based on priority/relevance, amount of patient data, type of patient data, and context of review of the patient data to develop a data model that determines how to optimize sizing and arrangement of the patient data based on these factors. In some embodiments, either of these data models can perform a utility-based analysis that factors cost of incorrectly displaying a subset of patient information relative to benefit of correctly displaying the subset of patient information.

In this regard, the machine learning component 502 can perform classifications, correlations, inferences and/or expressions associated with principles of artificial intelligence. For instance, the machine learning component 502 can employ an automatic classification system and/or an automatic classification. In one example, the machine learning component 502 can employ a probabilistic and/or statistical-based analysis (e.g., factoring into the analysis utilities and costs) to learn and/or generate inferences. The machine learning component 502 can employ any suitable machine-learning based techniques, statistical-based techniques and/or probabilistic-based techniques. For example, the machine learning component 502 can employ expert systems, fuzzy logic, SVMs, Hidden Markov Models (HMMs), greedy search algorithms, rule-based systems, Bayesian models (e.g., Bayesian networks), neural networks, other non-linear training techniques, data fusion, utility-based analytical systems, systems employing Bayesian models, etc. In another aspect, the machine learning component 502 can perform a set of machine learning computations. For example, the machine learning component 502 can perform a set of clustering machine learning computations, a set of logistic regression machine learning computations, a set of decision tree machine learning computations, a set of random forest machine learning computations, a set of regression tree machine learning computations, a set of least square machine learning computations, a set of instance-based machine learning computations, a set of regression machine learning computations, a set of support vector regression machine learning computations, a set of k-means machine learning computations, a set of spectral clustering machine learning computations, a set of rule learning machine learning computations, a set of Bayesian machine learning computations, a set of deep Boltzmann machine computations, a set of deep belief network computations, and/or a set of different machine learning computations.

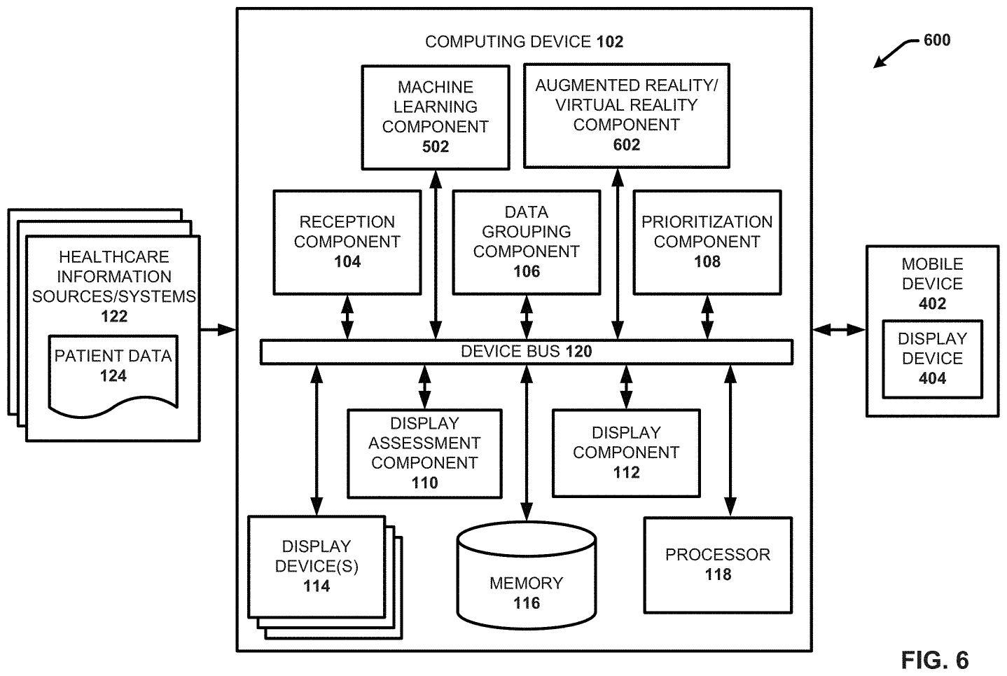

FIG. 6 illustrates a block diagram of another example, non-limiting system 600 that facilitates adapting the display of patient data based on display screen-real estate and patient monitoring context in accordance with one or more embodiments of the disclosed subject matter. System 600 includes same or similar features as system 500 with the addition of augmented reality/virtual reality component 602 (hereafter, AR/VR component 602). Repetitive description of like elements employed in respective embodiments is omitted for sake of brevity.

In some embodiments, the computing device can include an AR/VR component 602 to facilitate generating AR and/or VR visualizations in association with rendering the patient data. With these embodiments, the one or more display device 114 and/or the display device 404 can include an AR and/or VR device capable of rendering AR and/or VR representations. For example, as an AR device the display device (e.g., of the one or more display device 114 and/or display device 404) can be configured to render graphical elements as overlays onto actual (e.g., viewed through a transparent or partially transparent display worn as eyewear) view of an environment and/or views of an environment viewed generated from a camera or live video feed. With these embodiments the AR/VR component 602 can facilitate determining what components of the patient data 124 to render as overlay data and how to render the overlay data relative to the view of the environment (e.g., how to align the overlay data with one or more objects in the environment. For example, in one implementation, the display assessment component 110 can generate a view of a live video feed of a patient and/or healthcare facility environment for rendering via an AR display device. The display assessment component 110 can also determine relevant components of the patient data 124 to the entity viewing the patient data (e.g., for remote monitoring purposes) and direct the AR/VR component 602 to render the data as overly data onto the live view of the patient/healthcare facility. For example, in one implementation, the AR/VR component 602 can render information indicating a location on the patient's body associated with a clinically significant element, such as an area where pain is experienced, an area associated with fracture, lesion, tumor, etc. The overlay data can align with the patient body and include text and/or image data representing the clinically significant element (e.g., a red overly onto a portion of the body where swelling is observed).

The AR/VR component 602 can also facilitate rendering the patient data 124 in a virtual reality environment. With these embodiments, the display assessment component 110 and/or the AR/VR component 602 can configure and render representations of the patient data 124 in virtually limitless dimensions, expanding and configuring the patient data 124 in accordance with virtual display screen-real estate that appears much larger than one or more monitors. The display assessment component 110 however can still configure the size and arrangement of the patient data based on relevance/priority to the patient/medical reviewer context.

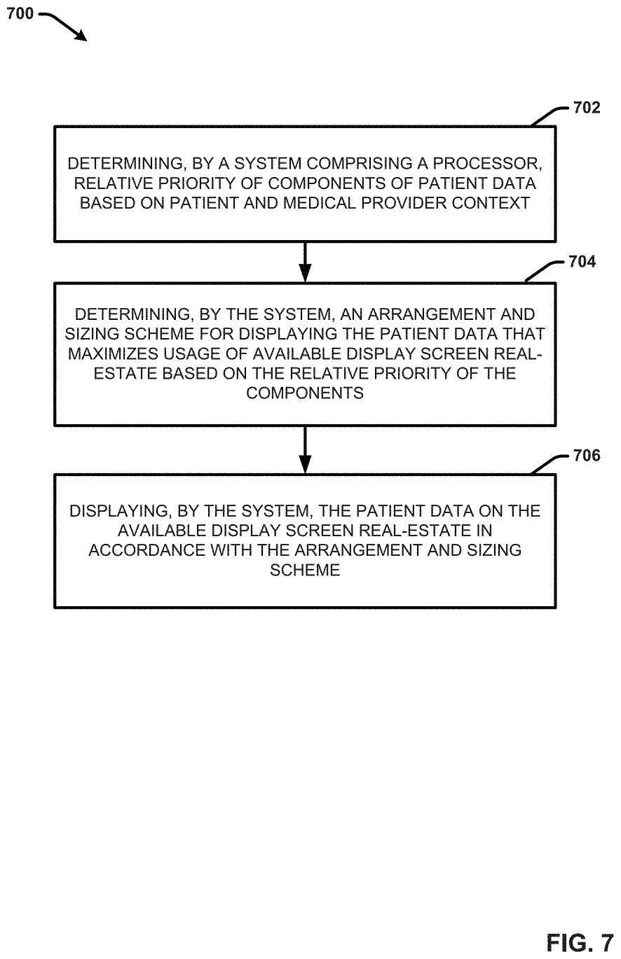

FIG. 7 provides a flow diagram of an example, non-limiting computer-implemented method 700 for adapting the display of patient data based on display screen-real estate and patient monitoring context in accordance with one or more embodiments of the disclosed subject matter. Repetitive description of like elements employed in respective embodiments is omitted for sake of brevity.

At 702, a system operatively coupled to a processor (e.g., system 100, system 400, system 500, system 600, or the like), can determine relative priority of components of patient data based on patient and medical provider context (e.g., via prioritization component 108). At 704 the system can determine an arrangement and sizing scheme for displaying the patient data that maximizes usage of available display screen real-estate based on the relative priority of the subsets (e.g., via display assessment component 110). At 706, the system can further display the patient data on the available display screen real-estate in accordance with the arrangement and sizing scheme (e.g., via display component 112).

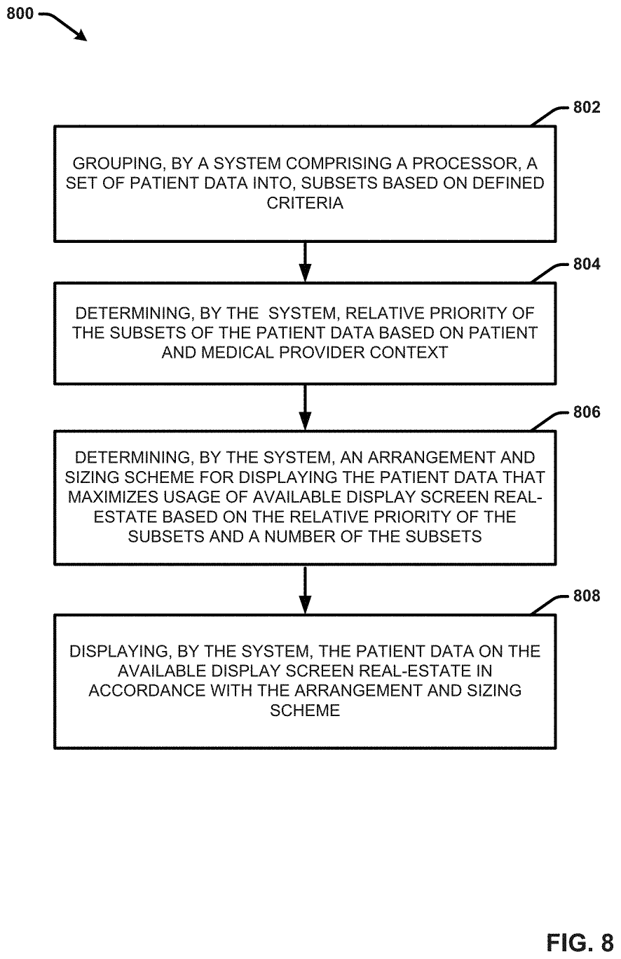

FIG. 8 provides a flow diagram of another example, non-limiting computer-implemented method 800 for adapting the display of patient data based on display screen-real estate and patient monitoring context in accordance with one or more embodiments of the disclosed subject matter. Repetitive description of like elements employed in respective embodiments is omitted for sake of brevity.

At 802, a system operatively coupled to a processor (e.g., system 100, system 400, system 500, system 600, or the like), can group a set of patient data into the subsets based on defined criteria (e.g., via the data grouping component 106). At 804, the system can determine relative priority of the subsets of the patient data based on patient and medical provider context (e.g., via prioritization component 108). At 806, the system can determine an arrangement and sizing scheme for displaying the patient data that maximizes usage of available display screen real-estate based on the relative priority of the subsets and a number of the subsets (e.g., via display assessment component 110). At 808, the system can further display the patient data on the available display screen real-estate in accordance with the arrangement and sizing scheme (e.g., via display component 112).

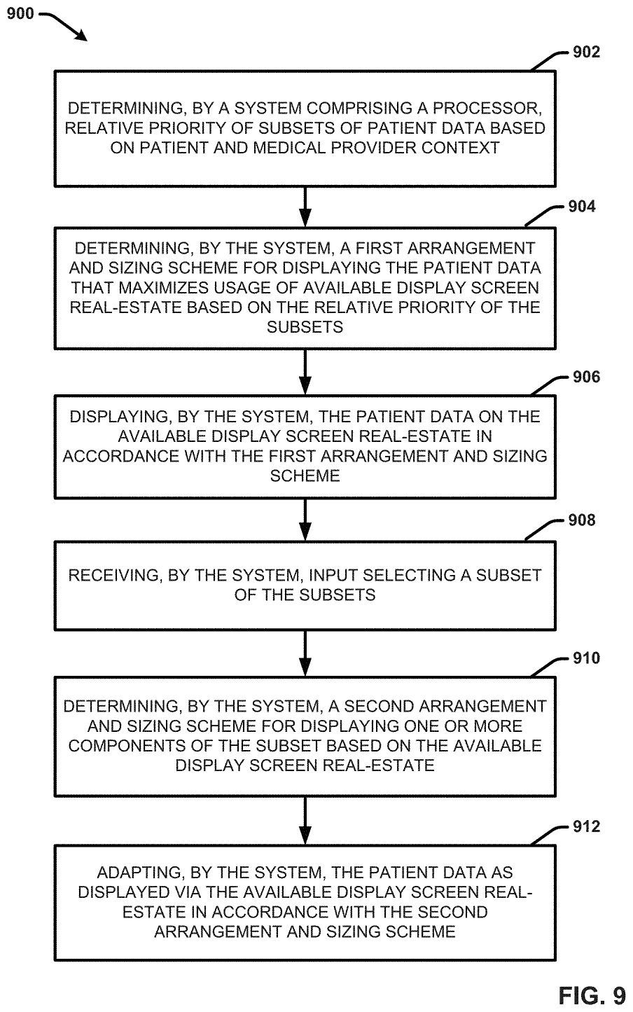

FIG. 9 provides a flow diagram of an example, non-limiting computer-implemented method 900 for adapting the display of patient data based on display screen-real estate and patient monitoring context in accordance with one or more embodiments of the disclosed subject matter. Repetitive description of like elements employed in respective embodiments is omitted for sake of brevity.

At 902, a system operatively coupled to a processor (e.g., system 100, system 400, system 500, system 600, or the like), can determine relative priority of subsets of patient data based on patient and medical provider context (e.g., via prioritization component 108). At 904, the system can determine a first arrangement and sizing scheme (e.g., a surveillance view) for displaying the patient data that maximizes usage of available display screen real-estate based on the relative priority of the subsets (e.g., via display assessment component 110). At 906, the system can further display the patient data on the available display screen real-estate in accordance with the first arrangement and sizing scheme (e.g., via display component 112). At 908, the system can receive input (e.g., via reception component 104) selecting a subset of the subsets (e.g., selecting a patient card from the surveillance view). At 910, the system can determine a second arrangement and sizing scheme for displaying one or more components of the subset based on the available display screen real-estate (e.g., via display assessment component 110). At 912, the system can adapt the patient data as displayed via the available display screen real-estate in accordance with the second arrangement and sizing scheme (e.g., via display component 112).

One or more embodiments can be a system, a method, and/or a computer program product at any possible technical detail level of integration. The computer program product can include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium can be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network can comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention can be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions can execute entirely on the user's computer, partly on the user's computer, as a stand-alone software package, partly on the user's computer and partly on a remote computer or entirely on the remote computer or server. In the latter scenario, the remote computer can be connected to the user's computer through any type of network, including a local area network (LAN) or a wide area network (WAN), or the connection can be made to an external computer (for example, through the Internet using an Internet Service Provider). In some embodiments, electronic circuitry including, for example, programmable logic circuitry, field-programmable gate arrays (FPGA), or programmable logic arrays (PLA) can execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It can be understood that each block of the flowchart illustrations and/or block diagrams, and combinations of blocks in the flowchart illustrations and/or block diagrams, can be implemented by computer readable program instructions.

These computer readable program instructions can be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions can also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions can also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams can represent a module, segment, or portion of instructions, which comprises one or more executable instructions for implementing the specified logical function(s). In some alternative implementations, the functions noted in the blocks can occur out of the order noted in the Figures. For example, two blocks shown in succession may, in fact, be executed substantially concurrently, or the blocks can sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

In connection with FIG. 10, the systems and processes described below can be embodied within hardware, such as a single integrated circuit (IC) chip, multiple ICs, an application specific integrated circuit (ASIC), or the like. Further, the order in which some or all of the process blocks appear in each process should not be deemed limiting. Rather, it should be understood that some of the process blocks can be executed in a variety of orders, not all of which can be explicitly illustrated herein.

With reference to FIG. 10, an example environment 1000 for implementing various aspects of the claimed subject matter includes a computer 1002. The computer 1002 includes a processing unit 1004, a system memory 1006, a codec 1035, and a system bus 1008. The system bus 1008 couples system components including, but not limited to, the system memory 1006 to the processing unit 1004. The processing unit 1004 can be any of various available processors. Dual microprocessors and other multiprocessor architectures also can be employed as the processing unit 1004.

The system bus 1008 can be any of several types of bus structure(s) including the memory bus or memory controller, a peripheral bus or external bus, or a local bus using any variety of available bus architectures including, but not limited to, Industrial Standard Architecture (ISA), Micro-Channel Architecture (MSA), Extended ISA (EISA), Intelligent Drive Electronics (IDE), VESA Local Bus (VLB), Peripheral Component Interconnect (PCI), Card Bus, Universal Serial Bus (USB), Advanced Graphics Port (AGP), Personal Computer Memory Card International Association bus (PCMCIA), Firewire (IEEE 1394), and Small Computer Systems Interface (SCSI).

The system memory 1006 includes volatile memory 1010 and non-volatile memory 1012, which can employ one or more of the disclosed memory architectures, in various embodiments. The basic input/output system (BIOS), containing the basic routines to transfer information between elements within the computer 1002, such as during start-up, is stored in non-volatile memory 1012. In addition, according to present innovations, codec 1035 can include at least one of an encoder or decoder, wherein the at least one of an encoder or decoder can consist of hardware, software, or a combination of hardware and software. Although, codec 1035 is depicted as a separate component, codec 1035 can be contained within non-volatile memory 1012. By way of illustration, and not limitation, non-volatile memory 1012 can include read only memory (ROM), programmable ROM (PROM), electrically programmable ROM (EPROM), electrically erasable programmable ROM (EEPROM), Flash memory, 3D Flash memory, or resistive memory such as resistive random access memory (RRAM). Non-volatile memory 1012 can employ one or more of the disclosed memory devices, in at least some embodiments. Moreover, non-volatile memory 1012 can be computer memory (e.g., physically integrated with computer 1002 or a mainboard thereof), or removable memory. Examples of suitable removable memory with which disclosed embodiments can be implemented can include a secure digital (SD) card, a compact Flash (CF) card, a universal serial bus (USB) memory stick, or the like. Volatile memory 1010 includes random access memory (RAM), which acts as external cache memory, and can also employ one or more disclosed memory devices in various embodiments. By way of illustration and not limitation, RAM is available in many forms such as static RAM (SRAM), dynamic RAM (DRAM), synchronous DRAM (SDRAM), double data rate SDRAM (DDR SDRAM), and enhanced SDRAM (ESDRAM) and so forth.

Computer 1002 can also include removable/non-removable, volatile/non-volatile computer storage medium. FIG. 10 illustrates, for example, disk storage 1014. Disk storage 1014 includes, but is not limited to, devices like a magnetic disk drive, solid state disk (SSD), flash memory card, or memory stick. In addition, disk storage 1014 can include storage medium separately or in combination with other storage medium including, but not limited to, an optical disk drive such as a compact disk ROM device (CD-ROM), CD recordable drive (CD-R Drive), CD rewritable drive (CD-RW Drive) or a digital versatile disk ROM drive (DVD-ROM). To facilitate connection of the disk storage 1014 to the system bus 1008, a removable or non-removable interface is typically used, such as interface 1016. It is appreciated that disk storage 1014 can store information related to a user. Such information might be stored at or provided to a server or to an application running on a user device. In one embodiment, the user can be notified (e.g., by way of output device(s) 1036) of the types of information that are stored to disk storage 1014 or transmitted to the server or application. The user can be provided the opportunity to opt-in or opt-out of having such information collected or shared with the server or application (e.g., by way of input from input device(s) 1028).

It is to be appreciated that FIG. 10 describes software that acts as an intermediary between users and the basic computer resources described in the suitable operating environment 1000. Such software includes an operating system 1018. Operating system 1018, which can be stored on disk storage 1014, acts to control and allocate resources of the computer 1002. Applications 1020 take advantage of the management of resources by operating system 1018 through program modules 1024, and program data 1026, such as the boot/shutdown transaction table and the like, stored either in system memory 1006 or on disk storage 1014. It is to be appreciated that the claimed subject matter can be implemented with various operating systems or combinations of operating systems.

A user enters commands or information into the computer 1002 through input device(s) 1028. Input devices 1028 include, but are not limited to, a pointing device such as a mouse, trackball, stylus, touch pad, keyboard, microphone, joystick, game pad, satellite dish, scanner, TV tuner card, digital camera, digital video camera, web camera, and the like. These and other input devices connect to the processing unit 1004 through the system bus 1008 via interface port(s) 1030. Interface port(s) 1030 include, for example, a serial port, a parallel port, a game port, and a universal serial bus (USB). Output device(s) 1036 use some of the same type of ports as input device(s) 1028. Thus, for example, a USB port can be used to provide input to computer 1002 and to output information from computer 1002 to an output device 1036. Output adapter 1034 is provided to illustrate that there are some output devices 1036 like monitors, speakers, and printers, among other output devices 1036, which require special adapters. The output adapters 1034 include, by way of illustration and not limitation, video and sound cards that provide a means of connection between the output device 1036 and the system bus 1008. It should be noted that other devices or systems of devices provide both input and output capabilities such as remote computer(s) 1038.

Computer 1002 can operate in a networked environment using logical connections to one or more remote computers, such as remote computer(s) 1038. The remote computer(s) 1038 can be a personal computer, a server, a router, a network PC, a workstation, a microprocessor based appliance, a peer device, a smart phone, a tablet, or other network node, and typically includes many of the elements described relative to computer 1002. For purposes of brevity, only a memory storage device 1040 is illustrated with remote computer(s) 1038. Remote computer(s) 1038 is logically connected to computer 1002 through a network interface 1042 and then connected via communication connection(s) 1044. Network interface 1042 encompasses wire or wireless communication networks such as local-area networks (LAN) and wide-area networks (WAN) and cellular networks. LAN technologies include Fiber Distributed Data Interface (FDDI), Copper Distributed Data Interface (CDDI), Ethernet, Token Ring and the like. WAN technologies include, but are not limited to, point-to-point links, circuit switching networks like Integrated Services Digital Networks (ISDN) and variations thereon, packet switching networks, and Digital Subscriber Lines (DSL).

Communication connection(s) 1044 refers to the hardware/software employed to connect the network interface 1042 to the bus 1008. While communication connection 1044 is shown for illustrative clarity inside computer 1002, it can also be external to computer 1002. The hardware/software necessary for connection to the network interface 1042 includes, for exemplary purposes only, internal and external technologies such as, modems including regular telephone grade modems, cable modems and DSL modems, ISDN adapters, and wired and wireless Ethernet cards, hubs, and routers.

While the subject matter has been described above in the general context of computer-executable instructions of a computer program product that runs on a computer and/or computers, those skilled in the art will recognize that this disclosure also can or can be implemented in combination with other program modules. Generally, program modules include routines, programs, components, data structures, etc. that perform particular tasks and/or implement particular abstract data types. Moreover, those skilled in the art will appreciate that the inventive computer-implemented methods can be practiced with other computer system configurations, including single-processor or multiprocessor computer systems, mini-computing devices, mainframe computers, as well as computers, hand-held computing devices (e.g., PDA, phone), microprocessor-based or programmable consumer or industrial electronics, and the like. The illustrated aspects can also be practiced in distributed computing environments where tasks are performed by remote processing devices that are linked through a communications network. However, some, if not all aspects of this disclosure can be practiced on stand-alone computers. In a distributed computing environment, program modules can be located in both local and remote memory storage devices.

As used in this application, the terms “component,” “system,” “platform,” “interface,” and the like, can refer to and/or can include a computer-related entity or an entity related to an operational machine with one or more specific functionalities. The entities disclosed herein can be either hardware, a combination of hardware and software, software, or software in execution. For example, a component can be, but is not limited to being, a process running on a processor, a processor, an object, an executable, a thread of execution, a program, and/or a computer. By way of illustration, both an application running on a server and the server can be a component. One or more components can reside within a process and/or thread of execution and a component can be localized on one computer and/or distributed between two or more computers. In another example, respective components can execute from various computer readable media having various data structures stored thereon. The components can communicate via local and/or remote processes such as in accordance with a signal having one or more data packets (e.g., data from one component interacting with another component in a local system, distributed system, and/or across a network such as the Internet with other systems via the signal). As another example, a component can be an apparatus with specific functionality provided by mechanical parts operated by electric or electronic circuitry, which is operated by a software or firmware application executed by a processor. In such a case, the processor can be internal or external to the apparatus and can execute at least a part of the software or firmware application. As yet another example, a component can be an apparatus that provides specific functionality through electronic components without mechanical parts, wherein the electronic components can include a processor or other means to execute software or firmware that confers at least in part the functionality of the electronic components. In an aspect, a component can emulate an electronic component via a virtual machine, e.g., within a cloud computing system.

In addition, the term “or” is intended to mean an inclusive “or” rather than an exclusive “or.” That is, unless specified otherwise, or clear from context, “X employs A or B” is intended to mean any of the natural inclusive permutations. That is, if X employs A; X employs B; or X employs both A and B, then “X employs A or B” is satisfied under any of the foregoing instances. Moreover, articles “a” and “an” as used in the subject specification and annexed drawings should generally be construed to mean “one or more” unless specified otherwise or clear from context to be directed to a singular form. As used herein, the terms “example” and/or “exemplary” are utilized to mean serving as an example, instance, or illustration and are intended to be non-limiting. For the avoidance of doubt, the subject matter disclosed herein is not limited by such examples. In addition, any aspect or design described herein as an “example” and/or “exemplary” is not necessarily to be construed as preferred or advantageous over other aspects or designs, nor is it meant to preclude equivalent exemplary structures and techniques known to those of ordinary skill in the art.

As it is employed in the subject specification, the term “processor” can refer to substantially any computing processing unit or device comprising, but not limited to, single-core processors; single-processors with software multithread execution capability; multi-core processors; multi-core processors with software multithread execution capability; multi-core processors with hardware multithread technology; parallel platforms; and parallel platforms with distributed shared memory. Additionally, a processor can refer to an integrated circuit, an application specific integrated circuit (ASIC), a digital signal processor (DSP), a field programmable gate array (FPGA), a programmable logic controller (PLC), a complex programmable logic device (CPLD), a discrete gate or transistor logic, discrete hardware components, or any combination thereof designed to perform the functions described herein. Further, processors can exploit nano-scale architectures such as, but not limited to, molecular and quantum-dot based transistors, switches and gates, in order to optimize space usage or enhance performance of user equipment. A processor can also be implemented as a combination of computing processing units. In this disclosure, terms such as “store,” “storage,” “data store,” data storage,” “database,” and substantially any other information storage component relevant to operation and functionality of a component are utilized to refer to “memory components,” entities embodied in a “memory,” or components comprising a memory. It is to be appreciated that memory and/or memory components described herein can be either volatile memory or nonvolatile memory, or can include both volatile and nonvolatile memory. By way of illustration, and not limitation, nonvolatile memory can include read only memory (ROM), programmable ROM (PROM), electrically programmable ROM (EPROM), electrically erasable ROM (EEPROM), flash memory, or nonvolatile random access memory (RAM) (e.g., ferroelectric RAM (FeRAM). Volatile memory can include RAM, which can act as external cache memory, for example. By way of illustration and not limitation, RAM is available in many forms such as synchronous RAM (SRAM), dynamic RAM (DRAM), synchronous DRAM (SDRAM), double data rate SDRAM (DDR SDRAM), enhanced SDRAM (ESDRAM), Synchlink DRAM (SLDRAM), direct Rambus RAM (DRRAM), direct Rambus dynamic RAM (DRDRAM), and Rambus dynamic RAM (RDRAM). Additionally, the disclosed memory components of systems or computer-implemented methods herein are intended to include, without being limited to including, these and any other suitable types of memory.

What has been described above include mere examples of systems and computer-implemented methods. It is, of course, not possible to describe every conceivable combination of components or computer-implemented methods for purposes of describing this disclosure, but one of ordinary skill in the art can recognize that many further combinations and permutations of this disclosure are possible. Furthermore, to the extent that the terms “includes,” “has,” “possesses,” and the like are used in the detailed description, claims, appendices and drawings such terms are intended to be inclusive in a manner similar to the term “comprising” as “comprising” is interpreted when employed as a transitional word in a claim. The descriptions of the various embodiments have been presented for purposes of illustration, but are not intended to be exhaustive or limited to the embodiments disclosed. Many modifications and variations can be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

What is claimed is:

1. A system that facilitates displaying patient information, comprising:
a memory that stores computer executable components; and
a processor that executes the computer executable components stored in the memory, wherein the computer executable components comprise:
a reception component that receives patient data;
a prioritization component that determines relative priority of subsets of the patient data to patient and medical provider context;
a display assessment component that determines an arrangement and sizing scheme for displaying the patient data based the relative priority of the subsets and available display screen real-estate; and
a display component that displays the patient data on the available display screen real-estate in accordance with the arrangement and sizing scheme.

2. The system of claim 1, wherein the display assessment component determines the arrangement and sizing scheme that maximizes usage of the available display screen real-estate.

3. The system of claim 1, wherein the computer executable components further comprise:
a data grouping component that groups the patient data into the subsets based on defined grouping criteria and wherein the display assessment component further determines the arrangement and sizing scheme based on a number of the subsets.

4. The system of claim 1, wherein the available display screen real-estate comprises a plurality of display monitors and wherein the arrangement and sizing scheme spreads the patient data across all of the plurality of display monitors.

5. The system of claim 1, wherein the display assessment component determines the arrangement and sizing scheme by sizing higher priority data elements of the patient data larger than lower priority data elements of the patient data.

6. The system of claim 1, wherein the arrangement and sizing scheme comprises a first arrangement and sizing scheme, and wherein based on reception of input selecting a subset of the subsets of patient data, the display assessment component determines a second arrangement and sizing scheme for displaying one or more components of the subset based on the available display screen real-estate, and wherein the display component adapts the patient data as displayed via the available display screen real-estate in accordance with the second arrangement and sizing scheme.

7. The system of claim 6, wherein the first arrangement and sizing scheme comprises a surveillance view comprising patient cards for different patients of a group of monitored patients represented in the patient data, wherein the subset corresponds to a patient card of the patient cards, and wherein the second arrangement and sizing scheme comprises a reduced size representation of the surveillance view on a portion of the display screen real-estate.

8. The system of claim 1, wherein the arrangement and sizing scheme comprises a first arrangement and sizing scheme, and wherein the display assessment component determines a second arrangement and sizing scheme that re-sizes or re-positions a subset of the patient data as displayed in accordance with the first arrangement and sizing scheme based on change in the patient or medical provider context, and wherein the display component adapts the patient data as displayed via the available display screen real-estate in accordance with the second arrangement and sizing scheme.

9. The system of claim 1, wherein the available display screen real-estate comprises a display of a mobile device and wherein the arrangement and sizing scheme distributes a subset of the patient data for displaying via the display of the mobile device.

10. The system of claim 9, wherein the display assessment component determines the subset based on role and privileges of an entity associated with the mobile device.

11. The system of claim 1, wherein the computer executable components further comprise:
a machine learning component that learns the patient data relative to patient and medical treater context and builds a data model.

12. The system of claim 11, wherein the data model performs a utility-based analysis that factors cost of incorrectly displaying a subset of patient information relative to benefit of correctly displaying the subset of patient information.

13. The system of claim 1, wherein the computer executable components further comprise:
an augmented reality component that displays a subset of the patient data as an overlay on eyewear of a medical provider.

14. A method comprising:
determining, by a system comprising a processor, relative priority of subsets of patient data based on patient and medical provider context;
determining, by the system, an arrangement and sizing scheme for displaying the patient data that maximizes usage of available display screen real-estate based on the relative priority of the subsets; and
displaying, by the system, the patient data on the available display screen real-estate in accordance with the arrangement and sizing scheme.

15. The method of claim 14, further comprising:
grouping, by the system, the patient data into the subsets based on defined criteria and further determining the arrangement and sizing scheme based on a number of the subsets.

16. The method of claim 14, wherein the determining the arrangement and sizing scheme comprises sizing higher priority components of the patient data larger than lower priority components of the patient data.

17. The method of claim 14, wherein the arrangement and sizing scheme comprises a first arrangement and sizing scheme, and wherein the method further comprises:
receiving, by the system, input selecting a subset of the subsets;
determining, by the system, a second arrangement and sizing scheme for displaying one or more components of the subset based on the available display screen real-estate; and
adapting, by the system, the patient data as displayed via the available display screen real-estate in accordance with the second arrangement and sizing scheme.

18. The method of claim 17, wherein the first arrangement and sizing scheme comprises a surveillance view comprising patient cards for different patients of a group of monitored patients represented in the patient data, wherein the subset corresponds to a patient card of the patient cards, and wherein the second arrangement and sizing scheme comprises a reduced size representation of the surveillance view on a portion of the display screen real-estate.

19. A machine-readable storage medium, comprising executable instructions that, when executed by a processor, facilitate performance of operations, comprising:
determining relative priority of subsets of patient data based on patient and medical provider context;
determining an arrangement and sizing scheme for displaying the patient data that maximizes usage of available display screen real-estate based on the relative priority of the subsets; and
displaying the patient data on the available display screen real-estate in accordance with the arrangement and sizing scheme.

20. The machine-readable storage medium of claim 19, wherein the available display screen real-estate comprises a plurality of display monitors and wherein the determining the arrangement and sizing scheme comprises:
grouping the patient data into logical groups based on the relative priority of the subsets and the number of the display monitors.
